(19) United States
(12) Patent Application Publication
(10) Pub. No.: US 2026/0083760 A1
(43) Pub. Date: Mar. 26, 2026

(54) STABLE LIQUID BORTEZOMIB FORMULATIONS

(71) Applicant: Riconpharma LLC, Denville, NJ (US)

(72) Inventors: Mukteeshwar Gande, Monroe, NJ (US); Praveen Reddy Billa, Flanders, NJ (US); SVB Janardhan Garikipati, Hyderabad (IN); Vinay Sudhindra Raichur, Hyderabad (IN); Vijaya Kumar Thota, Hyderabad (IN)

(73) Assignee: Riconpharma LLC, Denville, NJ (US)

(21) Appl. No.: 19/341,758

(22) Filed: Sep. 26, 2025

Related U.S. Application Data
(60) Provisional application No. 63/699,663, filed on Sep. 26, 2024.

(51) Int. Cl. A61K 31/69; A61K 9/00; A61K 9/08; A61K 47/02; A61K 47/26

(52) U.S. Cl. CPC A61K 31/69; A61K 9/0019; A61K 9/08; A61K 47/02; A61K 47/26

(57) ABSTRACT

Described herein are liquid compositions comprising: bortezomib; mannitol in an amount up to about 50 mg/mL; and a pharmaceutically acceptable carrier, wherein the liquid composition is for intravenous or subcutaneous use; and wherein the liquid composition comprises not more than (NMT) 1.0% of Impurity-E; Impurity-L; Impurity-J; Impurity-F; Impurity-I; or Impurity-U; and NMT than 3.0% of Total Impurities. Methods of making and using these compositions are also described herein.

### CROSS-REFERENCE TO RELATED APPLICATIONS

This application claimed the benefit of priority to U.S. Provisional Application No. 63/699,663, filed Sep. 26, 2024, the contents of which are hereby incorporated herein by reference in their entirety.

### FIELD OF THE DISCLOSURE

This invention relates to the liquid formulation of pharmaceutical compounds. More particularly, the invention relates to stable, pharmaceutically acceptable liquid compositions prepared from Bortezomib. The invention also relates to methods for preparing such compositions.

### BACKGROUND

Bortezomib is a proteasome inhibitor and it is indicated for the treatment of adult patients with multiple myeloma and mantle cell lymphoma. The currently available approved formulations in US are lyophilized powder of Bortezomib, where RLD is VELCADE with 3.5 mg/vial and Hospira has 505(b)(2) formulations of 1 mg/vial and 2.5 mg/vial. The powder which contains Bortezomib and mannitol needs to be reconstituted with 0.9% NaCl to a strength of 2.5 mg/mL and 1 mg/mL for subcutaneous and intravenous administration, respectively. There are other approved Generic versions of VELCADE available in the market, which are lyophilized products. There are also liquid bortezomib injections approved in US for only intravenous use, but all are discontinued.

The lyophilized formulations of Velcade were prepared by dissolving bortezomib and mannitol in solvent mixtures of water and tertiary butyl alcohol (TBA), and the solution was lyophilized to remove water and the solvents. U.S. Pat. No. 6,713,446B2 described the Velcade process and formation of bortezomib-mannitol ester which is important for stability of the formulation.

There are liquid bortezomib formulations prepared by utilizing different solubilizers.

U.S. Pat. No. 11,679,119B2 describes ready to use liquid formulation of bortezomib containing bortezomib and other pharmaceutical excipients such as mannitol, dimethyl sulfoxide, sodium acetate, water and, optionally, hydrochloric acid, sodium hydroxide, or a combination for pH adjustment. The composition is prepared by dissolving bortezomib into dimethyl sulfoxide as first solution and other excipients in water as second solution with pH in the range of 5 to 6.

U.S. Pat. No. 9,061,037B2 describes multi-dose liquid formulation of bortezomib with acidic buffer in which the composition contains non-aqueous solvent system suitable for injection i.e., Propylene glycol.

US20180110822A1 describes liquid formulation comprising Bortezomib and pharmaceutically acceptable excipients. The stable ready to use formulations consist of Bortezomib, chelating agents, solvents (ethanol, glycerine and water), Stabilizers (mannitol, sorbitol, arginine, glycine and lysine) and Anti-oxidants (monothioglycerol). The formulation uses excipients like monothioglycerol, arginine etc, which are not listed under IIG list for subcutaneous administration.

U.S. Pat. No. 8,263,578B2 describes similar to U.S. Pat. No. 9,061,037B2 with additional buffer having pH of 3.0 solvent system, where the buffer and the pH are selected such as to be effective to suppress formation of at least one of an amide degradation products when stored under specified storage conditions.

US20190290718A1 describes ready to dilute injectable formulation comprising 7.0 mg/mL to about 17.5 mg/mL bortezomib, non-aqueous solvent, pH adjusting agent and optionally an anti-oxidant. The amount of dissolved oxygen content in the invention is 10 ppm or less.

US20220133757A1 describes a composition comprising bortezomib, a polyhydric alcohol and water-soluble antioxidant. Specifically comprising bortezomib; mannitol; methionine; EDTA and glycine and sulfobutyl ether beta-cyclodextrin.

US20230062279A1 describes ready-to-use, aqueous composition of bortezomib with sufficient stability to allow its storage at a convenient temperature, preferably between 0° C. and 40° C., for a reasonable period of time. A sterile, ready-to-use, aqueous composition having 1 to 2.5 mg/ml of bortezomib, tonicity adjusting agent, a solvent or a cosolvent and a pH of from about 4.5 to about 6.5, wherein the tonicity adjusting agent is mannitol, the solvent is ethanol, the cosolvent is propylene glycol.

WO2017013208A1 describes Ready-to-use liquid pharmaceutical solution comprising an aqueous solvent; a Bortezomib ester contained in the solvent dissolved. Of which the bortezomib formation component is Mannitol and also contains NaCl and acetic acid/acetate buffer system giving a pH range of 3 to 6.

All the above-mentioned examples have limitations such as usage of non-aqueous solvents and utilization of new excipients which may not be acceptable to be administered by subcutaneous route.

### SUMMARY

A stable liquid formulation containing bortezomib was developed that can be suitable for subcutaneous and intravenous route. The liquid formulation was prepared by forming bortezomib-mannitol ester, thereby enhancing the solubility of bortezomib and stability of the product. The developed liquid formulation contains bortezomib along with bortezomib-mannitol ester in equilibrium with bortezomib. The bortezomib liquid formulation is a ready to use formulation and stable at storage temperature of 2° C. to 8° C.

The invention facilitates conversion of lyophilized formulation to a stable liquid formulation wherein the composition of the liquid formulation can be same as composition of VELCADE product post reconstitution, thereby eliminating any safety or efficacy challenges with the product for subcutaneous or intravenous routes. Along with the composition of the liquid formulation, the invention also describes manufacturing processes to achieve liquid formulation of bortezomib where bortezomib is dissolved in mannitol solutions at elevated temperatures. The conversion of bortezomib to bortezomib-mannitol ester can also be achieved by dissolving bortezomib and mannitol in water/organic solvent mixtures, thereby removing the organic solvent by a vacuum evaporation process.

### DETAILED DESCRIPTION

In the context of this invention “Bortezomib” refers to the pharmaceutically acceptable salts, solvates, hydrates and anhydrous forms thereof [(1R)-3-methyl-1-[[(2S)-1-oxo-3-phenyl-2-[(pyrazinylcarbonyl) amino]propyl]amino]butyl] boronic acid. The structure of Bortezomib is mentioned below:

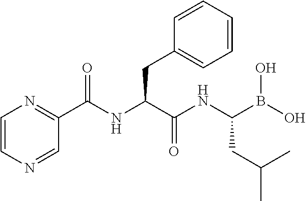 [SMILES: CC(C)C[C@H](NC(=O)[C@H](Cc1ccccc1)NC(=O)c1cnccn1)B(O)O]

As used herein, “liquid Bortezomib” or “ready to use Bortezomib” formulations refer to formulations that contain Bortezomib in dissolved or solubilised form and can be administered directly for subcutaneous application or diluted with the required IV diluent, preferably 0.9% NaCl solution, for administration via the intravenous route. The preferred concentration for subcutaneous administration is 2.5 mg/mL and for intravenous administration is 1 mg/mL.

Bortezomib is known to have limited solubility and stability in aqueous media. Therefore, it is supplied as a lyophilised formulation to improve the solubility and stability of the final formulation. The available marketed formulations are to be diluted with 0.9% NaCl solution before subcutaneous or intravenous administration.

Bortezomib forms boronic acid ester in presence of mannitol at a defined ratio which caters the increase of solubility and stability of Bortezomib in water. In the current invention mannitol and bortezomib containing compositions contain bortezomib ester and its respective hydrolysis product in equilibrium. The stable liquid formulation wherein the concentration of Bortezomib ranges from 1-2.5 mg/mL, more preferably 2.5 mg/mL. The concentration of mannitol ranges from 0-50 mg/mL, more preferably 25-50 mg/mL and most preferably 25 mg/mL. The concentration of sodium chloride ranges from 0-18 mg/mL, more preferably 9-18 mg/mL and most preferably 9 mg/mL.

The current invention discovers a suitable process for solubilizing bortezomib in aqueous solutions containing mannitol. The bortezomib is dissolved in mannitol solution at elevated temperature, which may facilitate formation of bortezomib-mannitol ester thereby increasing the solubility of bortezomib. The temperature for dissolution may be from about 25° C. to about 90° C. Bortezomib drug substance is available in different polymorphic, and more commonly available as form 1 or 2. Bortezomib form 1 has melting point of about 182° C. and bortezomib form 2 has melting point within 114-125° C. The preferred dissolution temperature utilizing bortezomib drug substance of form 1 is 60° C. to 90° C. and the preferred dissolution temperature utilizing bortezomib drug substance form 2 is 25° C. to 60° C. Another critical aspect of the invention includes control of dissolved oxygen levels preferably between 0-10 PPM and more preferably below 2 PPM before the addition of Bortezomib, during compounding and filling operations.

The current invention is a stable Bortezomib formulation as aqueous solution which is stable at 2-8° C. for intravenous or subcutaneous use. The formulations have a very low initial levels of degradants which are Impurity E and Impurity L, and are also controlled in stability at 2-8 C.°. The chemical names and the structures are presented below.

### Impurity E:

Chemical name: (S)—N-(1-amino-1-oxo-3-phenylpropan-2-yl)pyrazine-2-carboxamide
Structure:

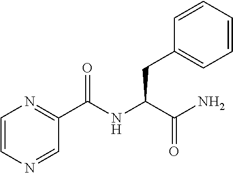 [SMILES: NC(=O)[C@H](Cc1ccccc1)NC(=O)c1cnccn1]

### Impurity F:

Chemical name: N—((S)-1-((R)-1-hydroxy-3-methylbutylamino)-1-oxo-3-phenylpropan-2-yl)pyrazine-2-carboxamide
Structure:

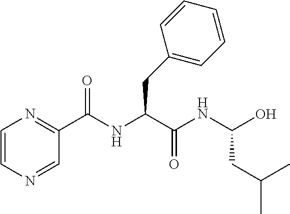 [SMILES: CC(C)C[C@@H](O)NC(=O)[C@H](Cc1ccccc1)NC(=O)c1cnccn1]

The manufacturing process to achieve the desired stable formulation is described in brief as follows:
i. Collect of approximately 90% of Batch volume of the of WFI in primary compounding vessel, at 20-25° C.
ii. Purging the filtered nitrogen into WFI till DO levels of <2 PPM are attained.
iii. Adding dispensed quantity of mannitol under continuous stirring to get clear solution.
iv. Adding dispensed quantity of Sodium chloride under continuous stirring to get clear solution.
v. Check and maintaining the DO levels of <2 PPM.
vi. Heating the solution to about 60-90 degree C.
vii. Adding dispensed quantity of Bortezomib API under continuous stirring and mixing till a clear solution is obtained.
viii. Cooling the solution to room temperature (20-25° C.)
ix. Making up the weight of the solution to 100% of batch size.
x. Filtration of solution
xi. Filling of solution into glass vials with Nitrogen headspace followed by stoppering and capping.

To summarize the stability data of one such formulation, it has Bortezomib content of 101.2% at initial stage which remained the same after 3 Months which indicates that the formulation retains the bortezomib content without any decrease. The degradation impurities including Impurity E, Impurity J, Impurity F, Impurity I, Impurity U, Impurity L were all found to be similar to initial degradation levels on storage at 2-8° C. The data indicates minimal degradation with the developed process.

Another aspect of the invention discloses suitable process for stable Bortezomib formulations which includes vacuum evaporation. The invention related to preparation of stable formulations by dissolving Bortezomib in non-aqueous solvent in one phase wherein the solvent can be any organic solvent which has high solubility of bortezomib, such as tertiary butyl alcohol, ethyl alcohol, etc. The second phase contains mannitol dissolved in water. Both the phases are then mixed and subjected to vacuum evaporation at suitable temperature of 30-70° C., more preferably 50° C. The concentrate obtained from the evaporation is then converted into liquid formulation with sodium chloride solution to obtain final formulation with concentration of Bortezomib ranges from 1-2.5 mg/mL, more preferably 2.5 mg/mL. The concentration of mannitol ranges from 0-50 mg/mL, more preferably 25-50 mg/mL and most preferably 25 mg/mL. The concentration of sodium chloride ranges from 0-18 mg/mL, more preferably 9-18 mg/mL and most preferably 9 mg/mL.

The manufacturing process to achieve the desired stable formulation is described in brief as follows:
i. Collect of approximately 55% of Batch volume of the of tertiary butyl alcohol in primary compounding vessel, at 20-25° C.
ii. Purging the filtered nitrogen into WFI till DO levels of <2 PPM are attained.
iii. Adding dispensed quantity of Bortezomib under continuous stirring to get clear solution.
iv. To another vessel, added 45% of Batch volume of water at 20-25° C.
v. Adding dispensed quantity of mannitol under continuous stirring to get clear solution.
vi. The solution of step v is added to non-aqueous phase (step i) and mixed thoroughly to form uniform solution
vii. Check and maintaining the DO levels of <2 PPM.
viii. The solution of step vii was subjected to rota evaporation at 50° C. and 300 mbar-60 mbar
ix. After, the residual solvent content was decreased below 3500 ppm, the assembly was discontinued from rota evaporation process and concentrate was discharged
x. The concentrate was then diluted with sodium chloride solution to obtain final desired concentration.
xi. Filtration of solution
xii. Filling of solution into glass vials with Nitrogen headspace followed by stoppering and capping.

To summarize the stability data of one such formulation, it has Bortezomib content of 97.9% at initial stage which remained the same after 3 Months which indicates that the formulation retains the bortezomib content without any decrease. The degradation impurities including Impurity J, Impurity E, Impurity F, Impurity I, Impurity U and Impurity L were all found to be similar to initial degradation levels after 3 months of storage at 2-8° C. The data indicates minimal degradation with the developed process.

### EXAMPLES

### Example 1: Preparation of Ready to Use Aqueous Solution of Bortezomib (Form 1) at 90° C.

Approximately 2.5 g of Mannitol was weighed and added into 90.0 g of water with continuous stirring. To the solution 0.9 g of weighed quantity of sodium chloride was added and stirred continuously to obtain a clear solution. The solution was purged with nitrogen to maintain the dissolved oxygen below 2 ppm. The solution was heated to a temperature of 90° C. To the heated solution, Bortezomib (Form 1) API, 0.25 g was added under continuous stirring to form a clear solution. The solution was cooled down to room temperature and final volume of the solution was made up to 100 g. All the operations were performed under sodium vapor lamp to minimize photo degradation. The solution was filtered through 0.2μ filter. 1.0 mL of solution was filled into each vial, stoppered with rubber stopper and sealed with flip off seals.

TABLE 1

| Condition | Specification | Initial |
|---|---|---|
| Assay | 90%-110% | 100.7 |
| Impurity-E | NMT 1.0% | 0.02 |
| Impurity-L | NMT 1.0% | 0.31 |
| Impurity-J | NMT 1.0% | 0.04 |
| Impurity-F | NMT 1.0% | 0.05 |
| Impurity-I | NMT 1.0% | ND |
| Impurity-U | NMT 1.0% | ND |
| Highest Unknown | NMT 0.5% | 0.01 |
| Total impurities | NMT 3.0% | 0.47 |

### Example 2: Preparation of Ready to Use Aqueous Solution of Bortezomib (Form 1) at 80° C.

Approximately 10.0 g of Mannitol was weighed and added into 360.0 g of water with continuous stirring. To the solution 3.6 g of weighed quantity of sodium chloride was added and stirred continuously to obtain a clear solution. The solution was purged with Nitrogen to maintain the dissolved oxygen below 2 ppm. The solution was heated to a temperature of 80° C. To the heated solution, Bortezomib (Form 1) API 1.00 g was added under continuous stirring to form a clear solution. The solution was cooled down to room temperature and final volume of the solution was made up to 400 g. All the operations were performed under sodium vapor lamp to minimize photo degradation. The solution was filtered through 0.2μ filter. 1.4 mL of solution was filled into each vial, stoppered with rubber stopper and sealed with flip off seals.

TABLE 2

| Condition | Specification | Initial | 2-8° C. (2 M) |
|---|---|---|---|
| Assay | 90%-110% | 100.8 | 102.1 |
| Impurity-E | NMT 1.0% | 0.02 | 0.07 |
| Impurity-L | NMT 1.0% | 0.17 | 0.38 |
| Impurity-J | NMT 1.0% | 0.03 | 0.08 |
| Impurity-F | NMT 1.0% | 0.05 | 0.08 |
| Impurity-I | NMT 1.0% | ND | 0.08 |
| Impurity-U | NMT 1.0% | ND | 0.01 |
| Highest Unknown | NMT 0.5% | 0.01 | 0.01 |
| Total impurities | NMT 3.0% | 0.31 | 0.78 |

### Example 3: Preparation of Ready to Use Aqueous Solution of Bortezomib (Form 1) at 72° C.

Approximately 10.0 g of Mannitol was weighed and added into 360.0 g of water with continuous stirring. To the solution 3.6 g of weighed quantity of sodium chloride was added and stirred continuously to obtain a clear solution. The solution was purged with Nitrogen to maintain the dissolved oxygen below 2 ppm. The solution was heated to a temperature of 72° C. To the heated solution, Bortezomib (Form 1) API 1.00 g was added under continuous stirring to form a clear solution. The solution was cooled down to room temperature and final volume of the solution was made up to 400 g. All the operations were performed under sodium vapor lamp to minimize photo degradation. The solution was filtered through 0.2μ filter. 1.4 mL of solution was filled into each vial, stoppered with rubber stopper and sealed with flip off seals.

### Example 4: Preparation of Ready to Use Aqueous Solution of Bortezomib (Form 1) at 60° C.

Approximately 5.00 g of Mannitol was weighed and added into 180.0 g of water with continuous stirring. To the solution 1.80 g of weighed quantity of sodium chloride was added and stirred continuously to obtain a clear solution. The solution was heated to a temperature of 60° C. To the heated solution, Bortezomib (Form 1) API 0.5 g was added under continuous stirring to form a clear solution. The solution was cooled down to room temperature and final volume of the solution was made up to 200 g. All the operations were performed under sodium vapor lamp to minimize photo degradation. The solution was filtered through 0.2μ filter.

TABLE 3

| Condition | Specification | Initial |
|---|---|---|
| Assay | 90%-110% | 97.8 |
| Impurity-E | NMT 1.0% | 0.02 |
| Impurity-L | NMT 1.0% | 0.23 |
| Impurity-J | NMT 1.0% | 0.06 |
| Impurity-F | NMT 1.0% | 0.08 |
| Impurity-I | NMT 1.0% | ND |
| Impurity-U | NMT 1.0% | ND |
| Highest Unknown | NMT 0.5% | 0.01 |
| Total impurities | NMT 3.0% | 0.45 |

### Example 5: Preparation of Ready to Use Aqueous Solution of Bortezomib (Form 2) at 50° C.

Approximately 2.50 g of Mannitol was weighed and added into 90.0 g of water with continuous stirring. To the solution 0.9 g of weighed quantity of sodium chloride was added and stirred continuously to obtain a clear solution. The solution was purged with Nitrogen to maintain the dissolved oxygen below 2 ppm. The solution was heated to a temperature of 50° C. To the heated solution, Bortezomib (Form 2) API 0.25 g was added under continuous stirring to form a clear solution. The solution was cooled down to room temperature and final volume of the solution was made up to 100 g. All the operations were performed under sodium vapor lamp to minimize photo degradation. The solution was filtered through 0.2μ filter. 1.4 mL of solution was filled into each vial, stoppered with rubber stopper and sealed with flip off seals.

TABLE 4

| Condition | Specification | Initial | 2-8° C. (2 week) |
|---|---|---|---|
| Assay | 90%-110% | 100.9 | 101.4 |
| Impurity-E | NMT 1.0% | 0.07 | 0.07 |
| Impurity-L | NMT 1.0% | 0.06 | 0.11 |
| Impurity-J | NMT 1.0% | 0.06 | 0.07 |
| Impurity-F | NMT 1.0% | 0.07 | 0.07 |
| Impurity-I | NMT 1.0% | ND | 0.03 |
| Impurity-U | NMT 1.0% | ND | ND |
| Highest Unknown | NMT 0.5% | 0.01 | 0.01 |
| Total impurities | NMT 3.0% | 0.35 | 0.39 |

### Example 6: Preparation of Ready to Use Aqueous Solution of Bortezomib (Form 2) at 40° C.

Approximately 2.50 g of Mannitol was weighed and added into 90.0 g of water with continuous stirring. To the solution 0.9 g of weighed quantity of sodium chloride was added and stirred continuously to obtain a clear solution. The solution was purged with Nitrogen to maintain the dissolved oxygen below 2 ppm. The solution was heated to a temperature of 40° C. To the heated solution, Bortezomib (Form 2) API 0.25 g was added under continuous stirring to form a clear solution. The solution was cooled down to room temperature and final volume of the solution was made up to 100 g. All the operations were performed under sodium vapor lamp to minimize photo degradation. The solution was filtered through 0.2μ filter. 1.4 mL of solution was filled into each vial, stoppered with rubber stopper and sealed with flip off seals.

TABLE 5

| Condition | Specification | Initial | 2-8° C. (2 week) |
|---|---|---|---|
| Assay | 90%-110% | 99.7 | 101.3 |
| Impurity-E | NMT 1.0% | 0.03 | 0.07 |
| Impurity-L | NMT 1.0% | 0.03 | 0.14 |
| Impurity-J | NMT 1.0% | 0.06 | 0.12 |
| Impurity-F | NMT 1.0% | 0.08 | 0.12 |
| Impurity-I | NMT 1.0% | 0.02 | 0.02 |
| Impurity-U | NMT 1.0% | ND | 0.01 |
| Highest Unknown | NMT 0.5% | 0.01 | 0.01 |
| Total impurities | NMT 3.0% | 0.23 | 0.53 |

### Example 7: Preparation of Ready to Use Aqueous Solution of Bortezomib (Form 2) at RT

Approximately 2.5 g of Mannitol was weighed and added into 90.0 g of water with continuous stirring. To the solution 0.9 g of weighed quantity of sodium chloride was added and stirred continuously to obtain a clear solution. The solution was purged with Nitrogen to maintain the dissolved oxygen below 2 ppm. To the solution, Bortezomib (Form 2) API 0.25 g was added under continuous stirring to form a clear solution. The solution was cooled down to room temperature and final volume of the solution was made up to 100 g. All the operations were performed under sodium vapor lamp to minimize photo degradation. The solution was filtered through 0.2μ filter.

TABLE 6

| Condition | Specification | Initial |
|---|---|---|
| Assay | 90%-110% | 98.3 |
| Impurity-E | NMT 1.0% | 0.03 |
| Impurity-L | NMT 1.0% | 0.02 |
| Impurity-J | NMT 1.0% | 0.05 |
| Impurity-F | NMT 1.0% | 0.09 |
| Impurity-I | NMT 1.0% | ND |
| Impurity-U | NMT 1.0% | ND |
| Highest Unknown | NMT 0.5% | 0.01 |
| Total impurities | NMT 3.0% | 0.21 |

### Example 8: Preparation of Ready to Use Aqueous Solution of Bortezomib (Form 1) at 80° C. Without Sodium Chloride

Approximately 2.5 g of Mannitol was weighed and added into 90.0 g of water with continuous stirring. The solution was heated to a temperature of 80° C. To the heated solution, Bortezomib (Form 1) API 0.25 g was added under continuous stirring to form a clear solution. The solution was cooled down to room temperature and final volume of the solution was made up to 100 g. The solution was filtered through 0.2μ filter. 1.0 mL of solution was filled into each vial, stoppered with rubber stopper and sealed with flip off seals.

TABLE 7

| Condition | Specification | Initial | 2-8° C. (3 M) |
|---|---|---|---|
| Assay | 90%-110% | 95.8 | 97.3 |
| Impurity-E | NMT 1.0% | 0.02 | 0.02 |
| Impurity-L | NMT 1.0% | 0.19 | 0.26 |
| Impurity-J | NMT 1.0% | 0.06 | 0.09 |
| Impurity-F | NMT 1.0% | 0.06 | 0.07 |
| Impurity-I | NMT 1.0% | ND | ND |
| Impurity-U | NMT 1.0% | ND | ND |
| Highest Unknown | NMT 0.5% | ND | ND |
| Total impurities | NMT 3.0% | 0.38 | 0.49 |

### Example 9: Preparation of Ready to Use Aqueous Solution of Bortezomib (Form 1) with Vacuum Evaporation Process with 0.9% Sodium Chloride

### Preparation of Concentrate:

Step 1: Approximately 330 g of water was taken and to it added 24.75 g of mannitol under continuous stirring to form a clear solution. The solution obtained was then purged with nitrogen to obtain DO level below 2%
Step 2: Approximately 171.6 g Tertiary butyl alcohol was taken and added 2.556 of Bortezomib was added under stirring at a temperature of 50° C. to obtain a clear solution.
Step 3: Both step 1 and step 2 were mixed together and charged for vacuum evaporation in Buchi Rotavap at vacuum of 300 mbar and gradually reduced to 150 mbar and then to 60 mbar.

### Preparation of Final Formulation:

Step 4: 207.65 g of concentrate was taken and made up to 1000 g. The batch was divided into two parts and to 1 part 0.9% sodium chloride was added. The solution was filtered through 0.2μ filter.

TABLE 8

| Condition | Specification | Initial | 2-8° C. (3 M) |
|---|---|---|---|
| Assay | 90%-110% | 97.9 | 97.5 |
| Impurity-E | NMT 1.0% | 0.01 | 0.01 |
| Impurity-L | NMT 1.0% | 0.03 | 0.08 |
| Impurity-J | NMT 1.0% | 0.06 | 0.11 |
| Impurity-F | NMT 1.0% | 0.10 | 0.12 |
| Impurity-I | NMT 1.0% | ND | ND |
| Impurity-U | NMT 1.0% | ND | ND |
| Highest Unknown | NMT 0.5% | 0.01 | 0.01 |
| Total impurities | NMT 3.0% | 0.20 | 0.32 |

### Example 10: Preparation of Ready to Use Aqueous Solution of Bortezomib (Form 1) with Vacuum Evaporation Process without Sodium Chloride

### Preparation of Concentrate:

Step 1: Approximately 57.798 g of water was taken and to it added 5.032 g of mannitol under continuous stirring to form a clear solution. The solution obtained was then purged with nitrogen to obtain DO level below 2%
Step 2: Approximately 32.002 g Tertiary butyl alcohol was taken and added 0.501 of Bortezomib was added under stirring at a at a temperature of 50° C. to obtain a clear solution.
Step 3: Both step 1 and step 2 were mixed together and charged for vacuum evaporation in Buchi Rotavap at vacuum of 300 mbar and gradually reduced to 150 mbar and then to 60 mbar.

### Preparation of Final Formulation:

Step 4: 10.014 g of concentrate was taken and made up to 24.879 g. The solution was filtered through 0.2μ filter.

TABLE 9

| Condition | Specification | Initial | 2-8° C. (3 M) |
|---|---|---|---|
| Assay | 90%-110% | 100.5 | 99.8 |
| Impurity-E | NMT 1.0% | 0.04 | 0.05 |
| Impurity-L | NMT 1.0% | 0.09 | 0.17 |
| Impurity-J | NMT 1.0% | 0.01 | 0.09 |
| Impurity-F | NMT 1.0% | 0.11 | 0.39 |
| Impurity-I | NMT 1.0% | ND | ND |
| Impurity-U | NMT 1.0% | ND | ND |
| Highest Unknown | NMT 0.5% | 0.01 | 0.01 |
| Total impurities | NMT 3.0% | 0.39 | 0.91 |

### Example 11: Preparation of Ready to Use Aqueous Solution of Bortezomib (Form 2) with Only Light Control and No DO Control at 40° C.

Approximately 5.00 g of Mannitol was weighed and added into 180.0 g of water with continuous stirring. To the solution 1.8 g of weighed quantity of sodium chloride was added and stirred continuously to obtain a clear solution. The solution was heated to a temperature of 40° C. To the heated solution, Bortezomib (Form 2) API 0.50 g was added under continuous stirring to form a clear solution. The solution was cooled down to room temperature and final volume of the solution was made up to 200 g. All the operations were performed under sodium vapor lamp. The solution was filtered through 0.2μ filter. 1.4 mL of solution was filled into each vial, stoppered with rubber stopper and sealed with flip off seals.

TABLE 10

| Condition | Specification | Initial |
|---|---|---|
| Assay | 90%-110% | 100.4 |
| Impurity-E | NMT 1.0% | 0.07 |
| Impurity-L | NMT 1.0% | 0.04 |
| Impurity-J | NMT 1.0% | 0.04 |
| Impurity-F | NMT 1.0% | 0.05 |
| Impurity-I | NMT 1.0% | 0.01 |
| Impurity-U | NMT 1.0% | 0.01 |
| Highest Unknown | NMT 0.5% | 0.01 |
| Total impurities | NMT 3.0% | 0.25 |

### Example 12: Preparation of Ready to Use Aqueous Solution of Bortezomib (Form 2) with Only DO Control and No Light Control at 40° C.

Approximately 5.00 g of Mannitol was weighed and added into 180.0 g of water with continuous stirring. To the solution 1.8 g of weighed quantity of sodium chloride was added and stirred continuously to obtain a clear solution. The solution was purged with Nitrogen to maintain the dissolved oxygen below 2 ppm. The solution was heated to a temperature of 40° C. To the heated solution, Bortezomib (Form 2) API 0.50 g was added under continuous stirring to form a clear solution. The solution was cooled down to room temperature and final volume of the solution was made up to 200 g. All the operations were performed under normal light. The solution was filtered through 0.2μ filter. 1.4 mL of solution was filled into each vial, stoppered with rubber stopper and sealed with flip off seals.

TABLE 11

| Condition | Specification | Initial |
|---|---|---|
| Assay | 90%-110% | 100.5 |
| Impurity-E | NMT 1.0% | 0.07 |
| Impurity-L | NMT 1.0% | 0.02 |
| Impurity-J | NMT 1.0% | 0.04 |
| Impurity-F | NMT 1.0% | 0.04 |
| Impurity-I | NMT 1.0% | 0.03 |
| Impurity-U | NMT 1.0% | 0.01 |
| Highest Unknown | NMT 0.5% | 0.01 |
| Total impurities | NMT 3.0% | 0.24 |

### Example 13: Preparation of Ready to Use Aqueous Solution of Bortezomib (Form 2) with Both DO Control and Light Control at 40° C.

Approximately 5.00 g of Mannitol was weighed and added into 180.0 g of water with continuous stirring. To the solution 1.8 g of weighed quantity of sodium chloride was added and stirred continuously to obtain a clear solution. The solution was purged with Nitrogen to maintain the dissolved oxygen below 2 ppm. The solution was heated to a temperature of 40° C. To the heated solution, Bortezomib (Form 2) API 0.50 g was added under continuous stirring to form a clear solution. The solution was cooled down to room temperature and final volume of the solution was made up to 200 g. All the operations were performed under sodium vapor lamp. The solution was filtered through 0.2μ filter. 1.4 mL of solution was filled into each vial, stoppered with rubber stopper and sealed with flip off seals.

TABLE 12

| Condition | Specification | Initial |
|---|---|---|
| Assay | 90%-110% | 101.9 |
| Impurity-E | NMT 1.0% | 0.07 |
| Impurity-L | NMT 1.0% | 0.04 |
| Impurity-J | NMT 1.0% | 0.04 |
| Impurity-F | NMT 1.0% | 0.06 |
| Impurity-I | NMT 1.0% | 0.03 |
| Impurity-U | NMT 1.0% | ND |
| Highest Unknown | NMT 0.5% | 0.01 |
| Total impurities | NMT 3.0% | 0.28 |

### Example 14: Dilution Data at 1 mg/mL

For intravenous infusion, the bortezomib injection has to be administered at a concentration of 1 mg/mL, for which aqueous solution of Bortezomib 2.5 mg/mL was diluted to 1 mg/mL with 0.9% sodium chloride injection.

TABLE 13

| | | BOF002-17 | VELCADE |
|---|---|---|---|
| Condition | Specification | Initial | (lot#12161999) |
| Assay | 90%-110% | 102.4 | 100 |
| Impurity-E | NMT 1.0% | 0.03 | 0.18 |
| Impurity-L | NMT 1.0% | 0.33 | 0.05 |
| Impurity-J | NMT 1.0% | 0.08 | 0.05 |
| Impurity-F | NMT 1.0% | 0.13 | 0.18 |
| Impurity-I | NMT 1.0% | ND | ND |
| Impurity-U | NMT 1.0% | ND | ND |
| Highest Unknown | NMT 0.5% | 0.01 | 0.01 |
| Total impurities | NMT 3.0% | 0.62 | 0.54 |

### Example 15: Comparison of Ester Content for 2.5 mg/mL and 1 mg/mL with RLD

### VELCADE Sample Preparation:

Reconstitute by adding 1.4 mL 0.9% NaCl and shake well. Take 90% of the reconstituted solution and add 10% D2O solution and analyse the proton NMR by using 400 mHZ.

### IH Sample Preparation:

Take 90% product solution and add 10% D2O solution and analyse the proton NMR by using 400 mHZ.

TABLE 14

| Batch. No | 2.5 mg/mL |
|---|---|
| Inhouse sample | 62.1 |
| US RLD- VELCADE | 67.7 |

What is claimed is:

1. A liquid composition comprising:
bortezomib;
mannitol in an amount up to about 50 mg/mL; and
a pharmaceutically acceptable carrier;
wherein the liquid composition is for intravenous or subcutaneous use; and
wherein the liquid composition comprises not more than (NMT) 1.0% of Impurity-E; Impurity-L; Impurity-J; Impurity-F; Impurity-I; or Impurity-U; and NMT than 3.0% of Total Impurities.

2. The liquid composition according to claim 1, wherein the liquid composition comprises NMT 0.5% of Impurity-E; Impurity-L; Impurity-J; Impurity-F; Impurity-I; or Impurity-U; and NMT than 1.0% of Total Impurities.

3. The liquid composition according to claim 1, wherein the bortezomib is present in an amount of from about 1 mg/mL to about 2.5 mg/mL.

4. The liquid composition according to claim 1, wherein the mannitol is present in an amount of from about 15 mg/mL to about 40 mg/mL.

5. The liquid composition according to claim 1, further comprising sodium chloride.

6. The liquid composition according to claim 5, wherein the sodium chloride is present in an amount up to about 18 mg/mL.

7. The liquid composition according to claim 6, wherein the sodium chloride is present in an amount of from about 5 mg/mL to about 15 mg/mL.

8. The liquid composition according to claim 6, wherein the sodium chloride is present in an amount of about 9 mg/mL.

9. A liquid composition comprising bortezomib, wherein the liquid composition is prepared by a process comprising:
a) dissolving an effective amount of bortezomib and mannitol in water/solvent mixtures;
b) evaporating the product of step a) to remove the solvent; and
c) diluting the product of step b) with water or sodium chloride to the desired bortezomib concentration.

10. The liquid composition according to claim 9, wherein the product of step c) comprises a bortezomib-mannitol ester.

11. The liquid composition according to claim 10, wherein the bortezomib-mannitol ester is in equilibrium with bortezomib.

12. The liquid composition according to claim 9, wherein the solvent comprises an organic solvent having a high solubility of bortezomib.

13. The liquid composition according to claim 12, wherein the solvent comprises a tertiary butyl alcohol or ethyl alcohol.

14. The liquid composition according to claim 9, wherein the concentration of bortezomib is from about 1 mg/mL to about 2.5 mg/mL.

15. The liquid composition according to claim 9, wherein the mannitol is present in an amount up to about 50 mg/mL.

16. The liquid composition according to claim 9, wherein the mannitol is present in an amount of from about 25 mg/mL to about 50 mg/mL.

17. The liquid composition process according to claim 9, wherein the sodium chloride in step c) is present in an amount up to about 18 mg/mL.

18. The liquid composition according to claim 17, wherein the sodium chloride in step c) is present in an amount of from about 9 mg/mL to about 18 mg/mL.

19. The liquid composition according to claim 9, further comprising a manufacturing vessel, wherein the headspace of the manufacturing vessel used for compounding and the finished product vial is maintained with a gas.

20. The liquid composition according to claim 19, wherein the dissolved oxygen levels are maintained between 0 to 10 PPM, before the addition of bortezomib, during compounding and filling operations.

21. A process for preparing liquid composition comprising bortezomib, wherein the process comprises:
dissolving bortezomib in the presence of mannitol at a temperature of from about 25° C. to about 90° C., wherein a bortezomib-mannitol ester is formed.

22. The process according to claim 21, wherein the bortezomib is dissolved at a temperature of from about 25° C. to about 60° C.; and wherein the bortezomib comprises polymorphic form 2.

23. The process according to claim 21, wherein the bortezomib is dissolved at a temperature of from about 60° C. to about 90° C.; and wherein the bortezomib comprises polymorphic form 1.

24. The process according to claim 21, wherein the bortezomib is present in an amount of from about 1 mg/mL to about 2.5 mg/mL.

25. The process according to claim 21, wherein the mannitol is present in an amount up to about 50 mg/mL.

26. The process according to claim 21, further comprising admixing sodium chloride.

27. The process according to claim 26, wherein the sodium chloride is present in an amount up to 18 mg/mL.

28. The process according to claim 21, wherein the headspace of the manufacturing vessel used for compounding and the finished product vial is maintained with nitrogen gas.

29. The process according to claim 28, wherein dissolved oxygen levels are maintained between 0 to 10 PPM, before the addition of bortezomib, during compounding and filling operations.

30. The process according to claim 29, wherein dissolved oxygen levels are maintained below 2 PPM.
